(19) United States
(12) Patent Application Publication
(10) Pub. No.: US 2009/0150081 A1
(43) Pub. Date: Jun. 11, 2009

(54) Automatic analyzer for enzyme immunoassays

(76) Inventor: Giuseppe Marcellino, Motta Sant'Anastasia (IT)

Correspondence Address: Modiano & Associati, Via Meravigli 16, 20123 Milan, IT

(21) Appl. No.: 11/989,245

(22) PCT Filed: Jul. 26, 2006
(86) PCT No.: PCT/EP2006/064687

§ 371 Date: Jan. 23, 2008

(30) Foreign Application Priority Data
Aug. 5, 2005 (IT) BO2005A000525

(51) Int. Cl. C12Q 1/25; G06F 19/00

(52) U.S. Cl. 702/19

(57) ABSTRACT

An automatic analyzer for enzyme immunoassays, of the type that comprises a serum dispensing assembly, a reagent dispensing assembly, an incubation assembly, a washing assembly, a reader assembly and a data processing and storage assembly. The analyzer comprises a unit for containing at least one well for performing analyses on a serum and at least one test-tube which contains a respective reagent; the unit is accommodated detachably within a respective internal frame of the analyzer. The reagent dispensing assembly, the incubation assembly, the washing assembly and the reader assembly can move from a first configuration of maximum distance from the unit accommodated in the frame to a second configuration of substantial overlap with at least one portion of the unit.

The present invention relates to an automatic analyzer for enzyme immunoassays.

### BACKGROUND OF THE INVENTION

Among enzyme immunoassays (EIA), one of the most widely used is the ELISA sandwich (an acronym which stands for “Enzyme-Linked ImmunoSorbent Assays”), which allows to detect and quantify substances such as peptides, proteins, antibodies and hormones.

In an ELISA test, an antigen must be immobilized on the surface of a solid. It is then treated with an antibody, which is generally associated with an enzyme. Detection is achieved by incubating this enzyme complex with a substrate which produces a detectable product. The fundamental element of the detection method is the extremely high specificity of the interaction between the antibody and the antigen.

ELISA assays are usually performed in microplatters provided with a large number of wells which passively bind antibodies and proteins.

The fact that the reagents of the ELISA are immobilized on the surface of the microplatter facilitates enormously the separation of the bound material from the unbound material during the assay. The possibility to wash away all the molecules that have bound nonspecifically makes ELISA a powerful means for measuring individual analytes in a raw extract.

ELISAs in which the antibodies are added in excess are also termed noncompetitive assays, in order to distinguish them from competitive EIAs, which provide for the simultaneous addition of antibodies or proteins in competition with each other.

Noncompetitive ELISA assays ensure a high sensitivity, since even with extremely low concentrations of analyte a large fraction thereof reacts with the excess antibody. However, they are less specific than competitive assays, although the use of monoclonal antibodies and of the so-called sandwich method has increased their specificity considerably.

The most commonly used enzymes are alkaline phosphatase (AP) and peroxidase; other enzymes such as b-galactosidase (bGal) are also used, albeit less widely.

Enzyme activity is measured with spectrophotometric measurement techniques.

Currently commercially available devices that perform this type of analysis have certain functional blocks: a serum dispensing block; a reagent dispensing block; an incubation block; a washing block; a reader block; a data storage and processing block.

The serum dispensing block generally comprises an arm provided with respective actuators for movement along the x, y and z axes and a metallic needle for dispensing into the wells of a microplatter (maximum 96 tests). Serum dispensing occurs by means of a dilution system composed of (at least) one syringe with a plunger tip made of polytetrafluoroethylene (PTFE, Teflon).

The reagent dispensing block utilizes the mechanical system for movement with x, y and z axes of the already-mentioned arm (a single arm is generally used both for sera and for reagents); the reagents are collected by means of an appropriately provided tip (generally but not exclusively made of a material such as plastics) with a process which uses (at least) one syringe with a plunger tip made of polytetrafluoroethylene (PTFE, Teflon). The reagents are then deposited in the respective reaction well of the microplatter, which is in its receptacle.

The incubation block is a system for heating the microplatters in order to bring the reaction to a predefined temperature according to the needs of the kit (from ambient temperature to a maximum of 37°). Said block can be already inserted in the support of the microplatters; otherwise, the apparatus must provide an element for the automated conveyance of the microplatters (this occurs in most cases in order to be able to have simultaneously both ambient-temperature incubation and 37°).

The washing block is composed of a suction pump, an intake pump and a washing head, which in turn is composed of at least 16 nozzles, eight for aspirating the reaction inside the wells and another eight for introducing therein the buffer solution for washing.

Therefore, this system generally entails that it is not possible to wash less than eight wells and that there is a mechanical system dedicated to the movement of the microplatter toward the washing head or vice versa.

The reader block is a photometric apparatus, which is composed of at least one lamp, a system with selective filters and at least one photodetector in order to measure the variation of the absorbance of the reaction in each individual well; it cannot be provided in the receptacle of the microplatters, because it must be kept in the dark (like a darkroom) in order to allow the resetting of the optical system and have no external interference. This block therefore necessarily requires a platter conveyance system.

Finally, the data processing and storage block is composed of a true central computer for loading and checking the data, step-by-step process control, downloading, processing and storage of the results.

Each of said blocks has problems that increase the complexity of use and compromise the precision and correctness of certain analyses performed with the apparatus. First of all, contaminations between one patient and another can occur in the serum dispensing block, since metallic needles are used which provide for washing with particular solutions; the management and execution of these washes entails a considerable cost increase for the apparatus.

In particular, the reagent dispensing block, despite having single-use tips, has a common outlet, which can become blocked easily; moreover, said outlet has metallic components (for example guides for the discharged tip) which can react with the small quantities of solution contained in the discharged tips (which are usually acid). Further, in view of the large number of patients that can be monitored simultaneously, the presence of large tanks is required in order to contain the reagents, with a consequent waste of reagent and a deterioration of its activity in future measurements.

The incubation block entails a movement of the microplatters in order to arrange them in its vicinity; the apparatus must therefore comprise suitable actuators, which are expensive and require periodic maintenance to avoid failures which lead to complete loss of functionality of the apparatus.

The washing block entails that washing is substantially simultaneous for all the wells, with a consequent waste of energy and of washing agents if a single well has been used (and therefore contaminated-dirtied).

The reading block provides for reading in a fixed point of the well: in this manner, an incorrect value may be obtained, since hormone growth can occur unevenly within the well.

Technical problems shared by all currently known devices are the inability to operate in partial mode in case of failures or breakages of certain parts: in fact, even if only one component is damaged, the apparatus is blocked, since the execution of processes occurs in parallel on all the equivalent components; if one of them fails, it cannot be isolated. Substantially, existing devices are scarcely versatile.

### SUMMARY OF THE INVENTION

The aim of the present invention is to obviate the above-mentioned drawbacks and meet the mentioned requirements, by providing an automatic analyzer for enzyme immunoassays that can operate in reduced mode in case of partial failure.

Within this aim, an object of the present invention is to avoid contaminations and pollution by eliminating the operations for washing the components and the associated costs therewith.

Another object of the present invention is to provide outlets which are difficult to block and are made of inert materials with respect to the reagents used.

Another object of the present invention is to allow the partial washing of the wells, leaving the unused ones unchanged.

A further object of the present invention is to read the wells in a nonpointwise manner, suitable for detecting uneven growths.

Another object of the present invention is to provide an automatic analyzer which is simple, relatively easy to provide in practice, safe in use, effective in operation, and has a relatively low cost.

This aim and these and other objects which will become better apparent hereinafter are achieved by the present automatic analyzer for enzyme immunoassays, of the type that comprises a serum dispensing assembly, a reagent dispensing assembly, an incubation assembly, a washing assembly, a reader assembly and a data processing and storage assembly, characterized in that it comprises a unit for containing at least one well for performing analyses on a serum and at least one test-tube which contains a respective reagent, said unit being accommodated detachably within a respective internal frame of said analyzer, said reagent dispensing assembly, said incubation assembly, said washing assembly and said reader assembly being movable from a first configuration of maximum distance from said unit accommodated in said frame to a second configuration of substantial overlap with at least one portion of said unit.

### BRIEF DESCRIPTION OF THE DRAWINGS

Further characteristics and advantages of the invention will become better apparent from the following detailed description of a preferred but not exclusive embodiment of an automatic analyzer for enzyme immunoassays, illustrated by way of non-limiting example in the accompanying drawings, wherein:

FIG. 1 is a schematic side view of an analyzer according to the invention;

FIG. 2 is a perspective view of a unit for containing at least one well for performing analyses on a serum of an analyzer according to the invention;

FIG. 3 is a schematic side view of the serum dispensing assembly of an analyzer according to the invention.

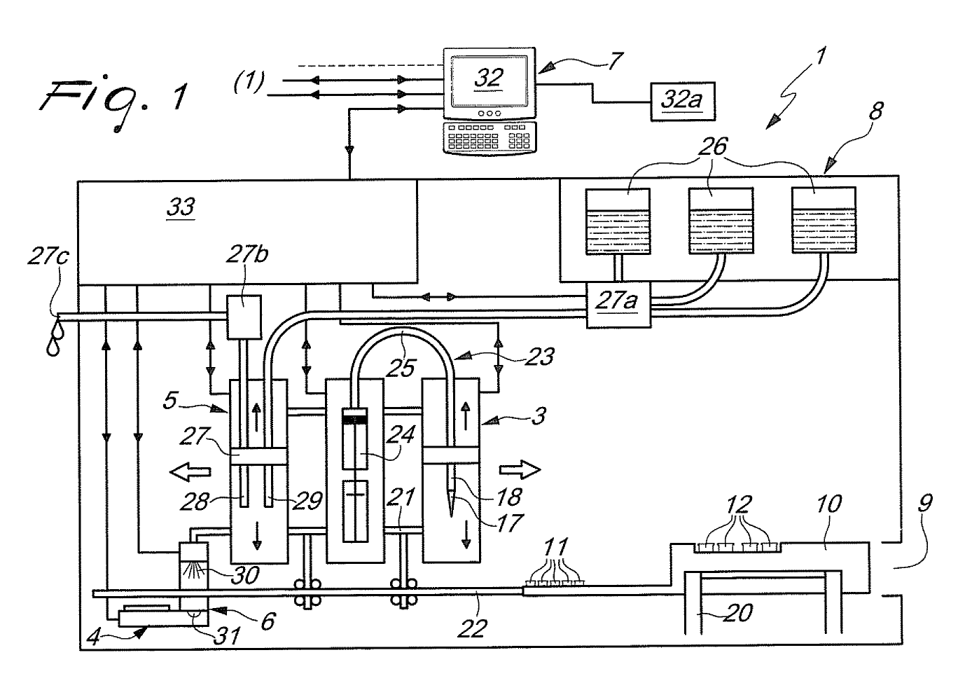

### DESCRIPTION OF THE PREFERRED EMBODIMENTS

With reference to the figures, the reference numeral 1 generally designates an automatic analyzer for enzyme immunoassays.

The analyzer 1 comprises a serum dispensing assembly 2, a reagent dispensing assembly 3, an incubation assembly 4, a washing assembly 5, a reader assembly 6 and a data processing and storage assembly 7.

The analyzer 1 is constituted by a box-like body 8, inside which said assemblies are installed. It is provided, at the front, with a slot 9 (which may optionally also be closed) for the insertion of a unit 10 for containing at least one well 11 for performing analyses on a serum and at least one test-tube 12 for containing a respective reagent.

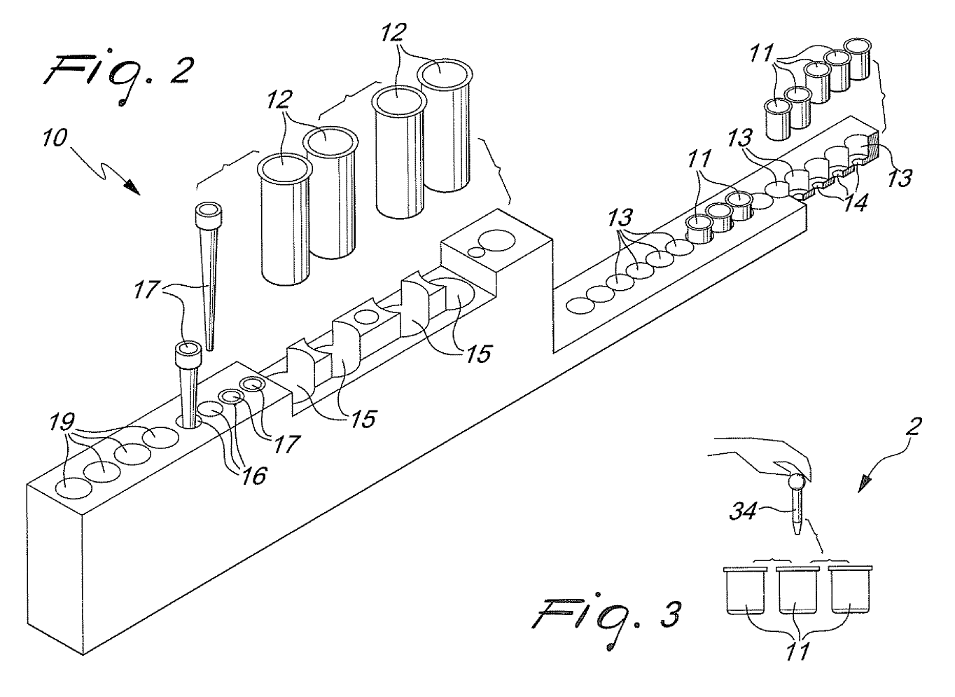

In the embodiment shown in FIG. 2, there are fifteen hollows 13, which are adapted to accommodate the wells 11 (they are in the end portion of the unit 10) and have a through hole 14 at their base (which has a smaller diameter than the well 11 that can be accommodated within the hollow 13), while four receptacles 15 are intended to accommodate the test-tubes 12 (they are in the initial portion of the unit 10).

The unit 10 has an elongated shape, since it is constituted substantially by two parallelepipeds, the end one of which (provided with the hollows 13) is shaped like a plate. The hollows 13 and the receptacles 15 are respectively aligned and substantially arranged at the centerline of the unit 10 on said parallelepipeds.

The initial portion of the unit 10 (the one provided with the receptacles 15) also comprises a series of pairs of holes; first holes 16 constitute the seat for the insertion of a respective tip 17 for a dispenser 18 of the reagent dispensing assembly 3, and second holes 19 constitute a discharge recess for every corresponding tip 17 after use.

The unit 10 is accommodated detachably within a respective internal frame 20 of the analyzer 1 in order to be coupled stably to the box-like body 8: the unit 10 has a plate made of ferromagnetic material (not shown in the figure) on its lower surface; said plate, when the unit 10 is accommodated within the frame 20, faces and lies proximate to an electromagnet, the actuation of which ensures the blocking of the unit.

The reagent dispensing assembly 3, the incubation assembly 4, the washing assembly 5 and the reader assembly 6 can move from a first configuration of maximum distance from the unit 10 (accommodated in the frame 20) to a second configuration for substantial overlap on at least one portion of the unit 10.

The reagent dispensing assembly 3, the incubation assembly 4, the washing assembly 5 and the reader assembly 6 are installed so that they can move on a common load-bearing structure 21, which can be moved substantially on a track 22 which is parallel to the unit 10 when it is accommodated within the internal frame 20.

The reagent dispensing assembly 3 comprises at least one pneumatic circuit 23, which is constituted by a cylinder with a corresponding piston 24, to the manifold of which a duct 25 is associated in which the opposite end is connected to the dispenser 18, which can be coupled to the single-use tips 17.

The reagent dispensing assembly 3 comprises a first actuator for translational motion along a substantially vertical axis of the dispenser 18 and a second actuator for moving the piston within the cylinder 24.

The incubation assembly 4 comprises at least one heat source, which can move from an inactive position to least one second position of alignment with at least one of the wells 11 as a consequence of the translational motion of the supporting structure 21 on the track 22.

The heat source has a maximum operating temperature of even more than 40° C.

The washing assembly 5 comprises a suction pump 27b, a pump 27a for introducing a buffer solution contained within respective containers 26, and a washing head 27, which is constituted by two nozzles 28, 29, of which one 28 is designed to aspirate the reaction into the well 11 and discharge it through a duct 27c externally, and the other one 29 is designed to introduce therein the buffer washing solution.

The reader assembly 6 comprises a light source 30, a filter and a scanning photodetector 31, which are rigidly coupled to the supporting structure 21.

The source 30 and the photodetector 31 are substantially mutually opposite and aligned and are separated by a space adapted for the passage of a portion of the unit that contains the well 11 being considered; the filter is interposed between the source 30 and the photodetector 31.

As a consequence of the translational motion of the supporting structure 21 on the track 22, the scanning photodetector 31 is activated to sense several times, detecting the absorbance value that corresponds to each point of the well 11 that is aligned with it.

The unit 10 is made of a material which is inert with respect to the reagents contained within the test-tubes 12.

The data processing and storage assembly 7 comprises a central computer 32 for control and management, which can be associated with a series of analyzers 1 and can be associated with suitable printing elements 32a.

Each analyzer 1 comprises a respective independent processor 33, which is interfaced with the central computer 32; the processor 33 operates even independently of the central computer 32.

The operating method of an analyzer 1 consists in providing a number of wells 11 equal to the number of patients whose respective serum is to be analyzed with a given test.

It is therefore necessary to dispense the serum of each patient into a respective well 11: this dispensing is performed manually (FIG. 3), by using a dosage device 34 provided with single-use interchangeable tips in order to avoid contamination.

The wells 11 that contain the serum must then be arranged on the unit 10, which also accommodates the test-tubes 12 that contain reagents in the amount sufficient to perform all the tests.

A number of single-use tips 17 must be then fitted on the unit for the dispenser 18 of the reagent dispensing assembly 3, in a number that is at least equal to the number of the test-tubes 12.

Once the initial preparation of the unit has been completed, it is necessary to insert it in the internal frame 20 of the analyzer 1, which must then be started.

Within the analyzer 1, the sera remain in incubation in the respective well 11 for a predefined period of time, also with the aid of the incubation assembly 4: the well 11 can optionally be lined internally with substances adapted to facilitate incubation.

Once incubation has occurred, it is necessary (if required by the test) to draw from one of the test-tubes 12, by means of the dispenser 18 with the single-use tip 17, at least one reagent and introduce it in a respective well 11.

It is therefore necessary to incubate again for a predefined period of time the serum and the reagent within the respective well 11.

Once incubation has occurred, the content of the well 11 must be washed and aspirated by means of the washing head 27 of the respective assembly 5.

Depending on the type of test being performed, it may be necessary to perform additional dispensings of reagents, which may even require corresponding incubations for predefined times and at the end of which it is advisable to perform suitable washes to prepare the well 11 for scanning.

When the wells 11 are correctly washed and the liquid that is present inside them has been aspirated completely, it is necessary to scan the surface of each well 11 by means of the reader assembly 6 in order to measure absorbance: said reading is performed by comparing the light signal detected by the photodetector 31 in various points of the well 11 (scanning) and by choosing among the values the one that is most significant (depending on the test being performed).

If the value of absorbance is known, it is possible to calculate the value related to the test performed by comparing this value with standard curves associated with the type of test being performed and with the reagents being used.

It has thus been shown that the invention achieves the proposed aim and objects.

The invention thus conceived is susceptible of numerous modifications and variations, all of which are within the scope of the appended claims.

All the details may further be replaced with other technically equivalent ones.

In the exemplary embodiments shown, individual characteristics, given in relation to specific examples, may actually be interchanged with other different characteristics that exist in other exemplary embodiments.

Moreover, it is noted that anything found to be already known during the patenting process is understood not to be claimed and to be the subject of a disclaimer.

In practice, the materials used, as well as the shapes and the dimensions, may be any according to requirements without thereby abandoning the scope of the protection of the appended claims.

The disclosures in Italian Patent Application No. BO2005A000525 from which this application claims priority are incorporated herein by reference.

What is claimed is:

1-17. (canceled)

18. An automatic analyzer for enzyme immunoassays, of the type that comprises a serum dispensing assembly, a reagent dispensing assembly, an incubation assembly, a washing assembly, a reader assembly and a data processing and storage assembly, wherein it comprises a unit for containing at least one well for performing analyses on a serum and at least one test-tube which contains a respective reagent, said unit being accommodated detachably within a respective internal frame of said analyzer, said reagent dispensing assembly, said incubation assembly, said washing assembly and said reader assembly being movable from a first configuration of maximum distance from said unit accommodated in said frame to a second configuration of substantial overlap with at least one portion of said unit.

19. The analyzer of claim 18, wherein said unit is elongated and comprises an initial parallelepipedal portion provided with a plurality of receptacles for test-tubes of reagent, which are substantially mutually aligned, and a plate-like end portion provided with several hollows for containing wells, which are likewise substantially aligned, said reagent test-tubes being substantially small.

20. The analyzer of claim 19, wherein said initial portion comprises a series of pairs of holes, the first hole constituting the seat for the insertion of a tip for the dispenser of the reagent dispensing assembly, the second hole constituting a recess for discharging said tip after use.

21. The analyzer of claim 19, wherein said hollows have their bottom affected by a through hole, which has a smaller diameter than said well and can be accommodated within said hollow.

22. The analyzer of claim 18, wherein said reagent dispensing assembly, said incubation assembly, said washing assembly and said reader assembly are installed so that they can move on a common supporting structure, which can perform substantially a translational motion on a rail, which is parallel to said unit, when said unit is accommodated in said internal frame.

23. The analyzer of claim 18, wherein said reagent dispensing assembly comprises at least one pneumatic circuit, which is constituted by a cylinder with a corresponding piston, with the manifold of which a duct is associated whose opposite end is connected to at least one dispenser which can be coupled to single-use tips.

24. The analyzer of claim 23, wherein said reagent dispensing assembly comprises a first actuator for translational motion along a substantially vertical axis of said dispenser and a second actuator for moving said piston within said cylinder.

25. The analyzer of claim 22, wherein said incubation assembly comprises at least one heat source, which can move from an inactive position to at least one second position for alignment with at least one of said wells, following the translational motion of said supporting structure on said rail.

26. The analyzer of claim 25, wherein said heat source has a maximum operating temperature of even more than 40° C.

27. The analyzer of claim 18, wherein said washing assembly comprises a suction pump, an intake pump and a washing head constituted by two nozzles, of which one is designed to aspirate the reaction into a well and the other one is designed to introduce therein a washing buffer solution.

28. The analyzer of claim 22, wherein said reader assembly comprises a light source, a filter and a scanning photodetector, which are rigidly coupled to said supporting structure, said source and said photodetector being substantially mutually opposite and aligned and separated by a space suitable for the passage of a portion of said unit which contains a well being considered, said filter being interposed between the source and the photodetector.

29. The analyzer of claim 18, wherein as a consequence of the translational motion of said supporting structure on said track, said scanning photodetector is activated for detection several times, detecting the absorbance value that corresponds to each point of the well that is aligned with it.

30. The analyzer of claim 18, wherein said unit is made of a material that is inert with respect to said reagents contained within the test-tubes.

31. The analyzer of claim 18, wherein said data processing and storage assembly comprises a central computer for control and management, which can also be associated with a series of analyzers which are mutually independent.

32. The analyzer of claim 31, wherein each one of said analyzers comprises a respective independent processor, which is interfaced with said central computer, said respective processor acting also independently of said central computer.

33. An operating method of an analyzer of claim 18, comprising the steps of:
preparing a number of wells equal to the number of patients whose respective serum is to be analyzed with a given test,
dispensing the serum of each patient within a respective well with a manual dispenser provided with a respective single use tip,
arranging on said unit said wells containing the serum,
arranging on said unit said test-tubes which contain reagents in a quantity sufficient to perform all the tests,
arranging on said units a number of single-use tips for said dispenser of said reagent dispensing assembly in a number that is at least equal to the number of said test-tubes,
inserting said unit within said internal frame of said analyzer,
starting said analyzer.

34. The method of claim 33, wherein the starting of said analyzer consists in:
incubating for a predefined period of time the serum within the respective well, also with the aid of the incubation assembly, said well being optionally lined internally with substances suitable to facilitate incubation,
drawing from said test-tube, by means of said dispenser, with a single-use tip, at least one reagent and introducing it in a respective well,
incubating for a predefined period of time the serum and the reagent within the respective well,
washing and aspirating the contents of said well by means of said washing head of the respective assembly,
performing optional additional dispensings of reagents, corresponding incubations and subsequent washes,
scanning said surface of said well by means of said reader assembly in order to measure absorbance,
calculating the value related to the test performed as a function of the determined absorbance value.
